(19) United States
(12) Patent Application Publication
(10) Pub. No.: US 2011/0160633 A1
(43) Pub. Date: Jun. 30, 2011

(54) METHOD FOR PRODUCING SHAPED POLYURETHANE FOAM WOUND DRESSINGS

(75) Inventors: Jan Schönberger, Haan (DE); Manfred Jordan, Lindenberg (DE); Michal Sabo, Lindenberg (DE); Bernd Mayer, Isny (DE)

(73) Assignee: Bayer MaterialScience AG, Leverkusen (DE)

(21) Appl. No.: 13/060,527

(22) PCT Filed: Aug. 17, 2009
(86) PCT No.: PCT/EP2009/006060

§ 371 Date: Feb. 24, 2011

(30) Foreign Application Priority Data
Aug. 27, 2008 (EP) 08163113.7

(51) Int. Cl. A61L 15/22; B29C 43/00; B32B 7/02

(52) U.S. Cl. 602/46; 264/321; 428/218

(57) ABSTRACT

The present invention relates to a process for producing shaped articles comprising, acting on a foam layer in a first region with a pressure of from 0 to 150 bar at a temperature of from 0° C. to less than or equal to 200° C., whereby the foam layer is compressed in the first region to greater than 12.5% to 100% of its original volume; acting on the foam layer in a second region with a pressure of from 50 to 150 bar at a temperature of from 100° C. to 200° C., whereby the foam layer is compressed in the second region from greater than 0% to 12.5% of its original volume; and wherein the foam layer comprises in the first region and the second region a polyurethane foam which is obtained by foaming and drying a composition comprising an aqueous, anionically hydrophilicized polyurethane dispersion.

The present invention relates to a process for producing shaped articles, where a foam layer is shaped in a particular region to a film. The foam layer includes a polyurethane foam which is obtained by foaming and drying a composition including an aqueous, anionically hydrophilicized polyurethane dispersion. It further relates to shaped articles produced by this process, and to the use thereof preferably as wound dressing.

It is possible in the management of wounds to employ wound dressings with a foam layer lying on the wound. This has proved to be advantageous because a climate which promotes healing can be achieved in the wound through the ability of the foam to absorb moisture emerging from the wound. When constructing such wound dressings it is expedient to provide also a sheet or film so that the actual foam is connected to this film. It is thus possible inter alia for the wound dressing to be stuck onto the skin (“island dressing”). The sheet connected to the foam, or the film, additionally represent a barrier to microbes (for example bacteria). In addition, the escape of wound fluid from the wound dressing is prevented. The film is ordinarily attached to the foam by bonding. However, pathogens are able to multiply at the junction between film and foam if wound fluid enters these regions. Furthermore, on exposure to moisture, such sheet- or film-foam assemblages may fail, meaning that the foam becomes detached on the moist wound from the backing material. Finally, the bonding of a film represents a further step in the production process, causing corresponding costs.

WO 2007/115696 A1, to which reference is made in its entirety, discloses a process for producing polyurethane foams for wound treatment, in which a composition comprising a polyurethane dispersion and specific coagulants is foamed and dried. The polyurethane dispersions can be obtained for example by preparing isocyanate-functional prepolymers from organic polyisocyanates and polymeric polyols having number average molecular weights from 400 g/mol to 8000 g/mol and OH functionalities of from 1.5 to 6 and, where appropriate, with hydroxy-functional compounds having molecular weights of from 62 g/mol to 399 g/mol and, where appropriate, isocyanate-reactive, anionic or potentially anionic and, where appropriate, nonionic hydrophilicizing agents. The free NCO groups of the prepolymer are then wholly or partly reacted where appropriate with amino-functional compounds having molecular weights of from 32 g/mol to 400 g/mol and with amino-functional, anionic or potentially anionic hydrophilicizing agents, with chain extension. The prepolymers are dispersed in water before, during or after the chain-extension step. Potentially ionic groups which are present where appropriate are converted by partial or complete reaction with a neutralizing agent into the ionic form.

GB 2 357 286 A discloses a process for producing a shaped polyurethane foam article for use as or in a wound dressing. The process includes the steps: provision of a last with a desired three-dimensional shape; application of an aqueous layer over the last; application of a layer of an isocyanate-terminated prepolymer over the last, with the prepolymer reacting with the aqueous layer on the last to form a polyurethane foam layer over the last; and stripping of the polyurethane foam layer off the last. The last is preferably hand-shaped, and the article is a burn glove. Shaped polyurethane foam articles obtainable from the process according to the invention are likewise provided. The polyurethane layer is typically 0.5 to 10 mm thick and has a density of 0.28 g/cm³ and an elongation at break of at least 150%. In this process, therefore, the foaming and setting of the polyurethane prepolymer is carried out in situ on a suitably shaped last. A disadvantage from the manufacturing viewpoint is, however, that a chemical reaction also occurs in the last step of the production of the article and takes time, requires suitable apparatuses and demands the safety measures necessary for chemical reactions.

WO 2001/00115 A2 discloses a shaped polyurethane article produced by crushing a polyurethane foam at elevated temperature for a preset time. It was found that by crushing a polyurethane foam into the desired shape, as is used for example for introduction into wound channels during a nose operation, followed by heating the polyurethane foam to an elevated temperature for a relatively short time. The polyurethane foam on cooling substantially retains its crushed shape but still remains substantially soft and pliable. The foams are preferably hydrophilic and flexible. Polyester- and/or polyether-polyurethanes are disclosed for the process. The foams disclosed therein are cylindrically compressed. It is not known how the foams behave on compression into other shapes, that is to say whether small radii of curvature are correctly reproduced for example in complex configurations. It is further unknown how the properties of the only generally described foam types are altered by the thermal compression.

There consequently remains a need for alternative shaped wound dressings with a foam layer and a film or sheet surrounding the foam layer, the intention being that no gaps occur therebetween. There is furthermore a need for a cost-effective production process for such wound dressings.

The invention therefore proposes a process for producing shaped articles,

where a pressure of from ≧0 bar to ≦150 bar acts on a foam layer in a first region at a temperature of from ≧0° C. to ≦200° C., and during the action of the pressure the foam layer is compressed in the first region to ≧12.5% to ≦100% of its original volume;

where a pressure of from ≧50 bar to ≦150 bar acts on a foam layer in a second region at a temperature of from ≧100° C. to ≦200° C., and during the action of the pressure the foam layer is compressed in the second region to ≧0% to ≦12.5% of its original volume; and

where the foam layer includes in the first region and the second region a polyurethane foam which is obtained by foaming and drying a composition including an aqueous, anionically hydrophilicized polyurethane dispersion (I).

A shaped article in the context of the present invention is produced by differential action of pressure and temperature on different regions of a foam layer. In this case, one region retains the characteristics of a foam, especially the ability to absorb fluid or permeability for water vapour, which is important for the use as wound dressing. Another region is changed in such a way that it is thermoformed to a film or sheet and thus acquires other properties. Owing to the fact that one foam layer has been used singly as starting material, the two regions are directly connected together and no gaps occur between the regions. It is also possible to refer to an integral island dressing in connection with the shaped article. In other words, therefore, the shaped article is in one-piece form in relation to the first and second region of the foam layer.

It is intended for the foam layer to include a foam which can be obtained from a foamed polyurethane dispersion. This foam layer is placed on the wound to be covered. This foam advantageously has a microporous, at least partly open-cell structure with intercommunicating cells.

The polyurethane dispersion (I) includes polyurethanes, with free isocyanate groups having been reacted at least in part with anionic or potentially anionic hydrophilicizing agents. Such hydrophilicizing agents are compounds which have functional groups reactive with isocyanate groups, such as amino, hydroxy or thiol groups, and in addition acidic groups or acid anion groups such as carboxylate, sulphonate or phosphonate groups.

After the foam layer has been dried it can advantageously be provided as flat roll goods. According to the invention, a pressure of from ≧0 bar to ≦150 bar acts on the foam layer in a first region at a temperature of from ≧0° C. to ≦200° C., and during the action of the pressure the foam layer is compressed in the first region to ≧12.5% to ≦100% of its original volume. The temperature may also be in a range from ≧20° C. to ≦100° C. or from ≧23° C. to ≦30° C. The pressure may also be in a range from ≧1 bar to ≦100 bar or from ≧2 bar to ≦50 bar. The compression can also be in a range from ≧25% to ≦90% or from ≧50% to ≦80% of the original volume. These pressures, temperatures and compressions retain the foam characteristics, so that this region can be placed on a wound. The pressure, temperature and compression acting on one volume element of the first region depends inter alia on the distance of the volume element from the edge of the recess of a mould or else on the shape of the mould itself if it has recesses. The foam layer may in this case likewise be thermoformed. For example, it may acquire a hemispherical shape or acquire indentations to enable better reception of parts of the body.

It is further provided for a pressure of from ≧50 bar to ≦150 bar to act on the foam layer in a second region at a temperature of from ≧100° C. to ≦200° C., and during the action of the pressure for the foam layer to be compressed in the second region to ≧0% to ≦12.5% of its original volume. The temperature may also be in a range from ≧120° C. to ≦190° C. or from ≧150° C. to ≦170° C. The pressure may also be in a range from ≧70 bar to ≦120 bar or from ≧90 bar to ≦110 bar. The compression may also be in a range from ≧5% to ≦10% or from ≧6% to ≦8% of the original volume. Under these conditions, the foam is compressed to a film or a sheet. It is then possible to apply for example adhesive for sticking the wound dressing onto the skin.

It is further possible for the film to be transparent or translucent, so that it is possible to observe the tissue surrounding the wound. It is possible to establish in this way whether an ulcer is spreading further or whether an unexpectedly large amount of exudate is escaping from the wound, without the need always to remove the wound dressing, and thus the risk of colonization of the wound by microbes is reduced. In addition, the need to change the wound dressings less often increases convenience for the patient and expedites the healing process.

The thermoforming can be carried out in suitable tools such as, for example, compression with dies and punches. However, in simple cases, the foam layer can also be provided with a curvature in a calender tool, in which case one region of the foam is not engaged by the calender rolls. Non-stick-coated tools are preferably used, it being possible to use both temporary non-stick coatings, for example by spraying on silicone oils, and corresponding permanent coatings such as, for example, Teflon or silica coatings, with preference for antistatic Teflon coatings in the case of a Teflon coating. The degree of compression can easily be adjusted in the thermoforming tool by providing an appropriate distance for example between die and punch or between calender rolls.

The process of the invention has the advantage that such an article with two regions can be produced from a single foam layer. This reduces the production costs. It is further favourable that a gap or join on the side facing the wound is avoided. Colonization by pathogens is avoided thereby and, in addition, a greater resistance to moisture of the wound dressing overall can be obtained.

In one embodiment of the process of the invention, the thermoforming of the second region is carried out for a period of from ≧45 seconds to ≦90 seconds. The thermoforming period may also be in a range from ≧50 seconds to ≦85 seconds or from ≧60 seconds to ≦80 seconds. By this is meant in general the time in which the second region of the foam layer is thermoformed by the action of pressure and heat. Thermoformed foam articles can be produced according to the invention also on a larger scale with the production cycle times according to the invention.

In a further embodiment of the process of the invention, the composition from which the polyurethane foam of the foam layer is obtained further includes additives which are selected from the group including fatty acid amides, sulphosuccinamides, hydrocarbonsulphonates, hydrocarbon sulphates, fatty acid salts, alkyl polyglycosides and/or ethylene oxide/propylene oxide block copolymers.

Additives of this type can act as foam formers and/or foam stabilizers. The lipophilic radical in the fatty acid amides, sulphosuccinamides, hydrocarbonsulphonates, hydrocarbon sulphates or fatty acid salts preferably comprises ≧12 to ≦24 carbon atoms. Suitable alkyl polyglycosides are obtainable for example by reacting long-chain monoalcohols (≧4 to ≦22 C atoms in the alkyl radical) with mono-, di- or polysaccharides. Also suitable are alkylbenzenesulphonates or alkylbenzene sulphates having ≧14 to ≦24 carbon atoms in the hydrocarbon radical.

The fatty acid amides are preferably those based on mono- or di-(C₂/C₃-alkanol)amines. The fatty acid salts may be for example alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coconut fatty acid, tallow fatty acid, soya fatty acid and the hydrogenation products thereof

Foam stabilizers which can be used by way of example are mixtures of sulphosuccinamides and ammonium stearates, these comprising preferably ≧20% by weight to ≦60% by weight, particularly preferably ≧30% by weight to ≦50% by weight of ammonium stearates and preferably ≧40% by weight to ≦80% by weight, particularly preferably ≧50% by weight to ≧70% by weight of sulphosuccinamides.

Further foam stabilizers which can be used by way of example are mixtures of fatty alcohol polyglycosides and ammonium stearates, these comprising preferably ≧20% by weight to ≦60% by weight, particularly preferably ≧30% by weight to ≦50% by weight of ammonium stearates and preferably ≧40% by weight to ≦80% by weight, particularly preferably ≧50% by weight to ≦70% by weight of fatty alcohol polyglycosides.

The ethylene oxide/propylene oxide block copolymers are adducts of ethylene oxide and propylene oxide onto OH- or NH-functional starter molecules.

Suitable starter molecules in principle are inter alia water, polyethylene glycols, polypropylene glycols, glycerol, trimethylolpropane, pentaerythritol, ethylenediamine, tolylenediamine, sorbitol, sucrose and mixtures thereof.

Starters preferably employed are di- or trifunctional compounds of the aforementioned type. Polyethylene glycol or polypropylene glycol are particularly preferred.

Block copolymers differing in type can be obtained through the respective amount of alkylene oxide and the number of ethylene oxide (EO) and propylene oxide (PO) blocks.

It is also possible in principle for the copolymers which are per se composed strictly blockwise of ethylene oxide or propylene oxide also to have mixed blocks of EO and PO.

Such mixed blocks are obtained if mixtures of EO and PO are employed in the polyaddition reaction so that, based on this block, a random distribution of EO and PO in this block results.

The EO/PO block copolymers employed according to the invention preferably have contents of ethylene oxide units of ≧5% by weight, particularly preferably ≧20% by weight and very particularly preferably ≧40% by weight based on the total of the ethylene oxide and propylene oxide units present in the copolymer.

The EO/PO block copolymers employed according to the invention preferably have contents of ethylene oxide units of ≦95% by weight, particularly preferably ≧90% by weight and very particularly preferably ≦85% by weight based on the total of the ethylene oxide and propylene oxide units present in the copolymer.

The EO/PO block copolymers employed according to the invention preferably have number average molecular weights of ≧1000 g/mol, particularly preferably ≧2000 g/mol, very particularly preferably ≧5000 g/mol.

The EO/PO block copolymers employed according to the invention preferably have number average molecular weights of ≦10 000 g/mol, particularly preferably ≦9500 g/mol, very particularly preferably ≦9000 g/mol.

One advantage of the use of the EO/PO block copolymers is that the resulting foam has a lower hydrophobicity than on use of other stabilizers. It is possible thereby to have a favourable effect on the absorption behaviour for fluids. In addition, non-cytotoxic foams are obtained on use of the EO/PO block copolymers in contrast to other stabilizers.

It is possible for the ethylene oxide/propylene oxide block copolymers to have a structure according to general formula (1):

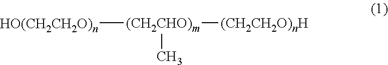

where the value for n is in a range from ≧2 to ≦200, and the value for m is in a range from ≧10 to ≦60.

EO/PO block copolymers of the aforementioned type are particularly preferred where they have a hydrophilic-lipophilic balance (HLB) of ≧4, particularly preferably of ≧8 and very particularly preferably of ≧14. The HLB is calculated by the formula HLB=20·Mh/M, where Mh is the number average molecular mass of the hydrophilic portion of the molecule formed from ethylene oxide, and M is the number average molecular mass of the whole molecule (Griffin, W. C.: Classification of surface active agents by HLB, J. Soc. Cosmet. Chem. 1, 1949). However, the HLB is ≦19, preferably ≦18.

In one embodiment of the process of the invention, the aqueous, anionically hydrophilicized polyurethane dispersion (I) is obtainable by

A) providing isocyanate-functional prepolymers which are obtainable from a reaction mixture including
A1) organic polyisocyanates and
   A2) polymeric polyols having number average molecular weights of from ≧400 g/mol to ≦8000 g/mol and OH functionalities of from ≧1.5 to ≦6
and where subsequently

B) the free NCO groups of the prepolymers are wholly or partly reacted with
B1) amino-functional, anionic or potentially anionic hydrophilicizing agents,

with chain extension, and the prepolymers are dispersed in water before, during or after step B), and where potentially ionic groups present in the reaction mixture are converted by partial or complete reaction with a neutralizing agent into the ionic form.

Preferred aqueous, anionic polyurethane dispersions (I) have a low degree of hydrophilic anionic groups, preferably from ≧0.1 to ≦15 milliequivalents per 100 g of solid resin.

In order to achieve good sedimentation stability, the number average particle size of the specific polyurethane dispersions is preferably ≦750 nm, particularly preferably ≦500 nm, determined by laser correlation spectroscopy.

The ratio of NCO groups in the compounds of component A1) to NCO-reactive groups such as amino, hydroxy or thiol groups in the compounds of components A2) to A4) in the production of the NCO-functional prepolymer is from ≧1.05 to ≦3.5, preferably ≧1.2 to ≦3.0, particularly preferably ≧1.3 to ≦2.5.

The amino-functional compounds in stage B) are employed in an amount such that the equivalent ratio of isocyanate-reactive amino groups in these compounds to the free isocyanate groups in the prepolymer is from ≧40% to ≦150%, preferably between ≧50% and ≦125%, particularly preferably between ≧60% and ≦120%.

Suitable polyisocyanates of component A1) are aromatic, araliphatic, aliphatic or cycloaliphatic polyisocyanates having an NCO functionality of ≧2.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any isomer content, 1,4-cyclohexylene diisocyanate, 1,4-phenylene diisocyanate, 2,4- and/or 2,6-tolylene diisocyanate (TDI), 1,5-naphthylene diisocyanate, 2,2′- and/or 2,4′- and/or 4,4′-diphenylmethane diisocyanate (MDI), 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) having C₁- to C₈-alkyl groups.

Besides the aforementioned polyisocyanates it is also possible to employ proportions of modified diisocyanates having a uretdione, isocyanurate, urethane, allophanate, biuret, iminooxadiazinedione and/or oxadiazinetrione structure, and unmodified polyisocyanate having more than 2 NCO groups per molecule, such as, for example, 4-isocyanatomethyl-1,8-octane diisocyanate (nonane triisocyanate) or triphenylmethane 4,4′,4″-triisocyanate.

Preferred polyisocyanates or polyisocyanate mixtures of the aforementioned type preferably have exclusively aliphatically and/or cycloaliphatically bound isocyanate groups and an average NCO functionality of the mixture of from ≧2 to ≦4, preferably ≧2 to ≦2.6 and particularly preferably ≧2 to ≦2.4.

It is particularly preferred to employ in A1) 1,6-hexamethylene diisocyanate, isophorone diisocyanate, the isomeric bis(4,4′-isocyanatocyclohexyl)methanes, and mixtures thereof.

The polymeric polyols employed in A2) have a number average molecular weight Mn of from ≧400 g/mol to ≦8000 g/mol, preferably from ≧400 g/mol to ≦6000 g/mol and particularly preferably from ≧600 g/mol to ≦3000 g/mol. They preferably have an OH functionality of from ≧1.5 to ≦6, particularly preferably from ≧1.8 to ≦3, very particularly preferably from ≧1.9 to ≦2.1.

Examples of such polymeric polyols are polyester polyols, polyacrylic polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be employed in A2), singly or in any mixtures with one another.

Such polyester polyols are polycondensates of diols, and where appropriate triols and tetraols, and dicarboxylic acids, and where appropriate tricarboxylic acids and tetracarboxylic acids or hydroxy carboxylic acids or lactones. Instead of the free polycarboxylic acids it is also possible to use the corresponding polycarboxylic anhydrides or corresponding polycarboxylic esters of lower alcohols to prepare the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or hydroxypivalic acid neopentyl glycol ester, with preference for hexanediol(1,6) and isomers, neopentyl glycol and hydroxypivalic acid neopenthyl glycol ester. Besides these, it is also possible to employ polyols such as trimethylolpropane, glycerol, erythritol, pentaerythritol, triemthylolbenzene or trishydroxyethyl isocyanurate.

Dicarboxylic acids which can be employed are phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. The corresponding anhydrides can also be used as source of acid.

If the average functionality of the polyol to be esterified is ≧2, it is possible in addition also to use monocarboxylic acids such as benzoic acid and hexanecarboxylic acid.

Preferred acids are aliphatic or aromatic acids of the aforementioned type. Adipic acid, isophthalic acid and, where appropriate, trimellitic acid are particularly preferred.

Hydroxy carboxylic acids which can be used as participants in the reaction to prepare a polyester polyol with terminal hydroxyl groups are for example hydroxycaproic acid, hydroxybutyric acid, hydroxydecanoic acid, hydroxystearic acid and the like. Suitable lactones are caprolactone, butyrolactone and homologues. Caprolactone is preferred.

It is likewise possible to employ in A2) polycarbonates having hydroxyl groups, preferably polycarbonate diols, having number average molecular weights Mn of from ≧400 g/mol to ≦8000 g/mol, preferably ≧600 g/mol to ≦3000 g/mol. These are obtainable by reacting carbonic acid derivatives such as diphenyl carbonate, dimethyl carbonate or phosgene with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the aforementioned type.

The polycarbonate diol preferably comprises ≧40% by weight to ≦100% by weight of hexanediol, preferably 1,6-hexanediol and/or hexanediol derivatives. Such hexanediol derivatives are based on hexanediol and, besides terminal OH groups, also have ester or ether groups. Such derivatives are obtainable by reacting hexanediol with excess caprolactone or by self-etherification of hexanediol to give dihexylene or trihexylene glycol.

Instead of or in addition to pure polycarbonate diols it is also possible to employ polyether-polycarbonate diols in A2).

The polycarbonates having hydroxyl groups preferably have a linear structure.

It is likewise possible to employ polyether polyols in A2).

Suitable examples are polytetramethylene glycol polyethers like those obtainable by polymerizing tetrahydrofuran by means of cationic ring opening.

Likewise suitable polyether polyols are the adducts of styrene oxide, ethylene oxide, propylene oxide, butylene oxide and/or epichlorohydrin with di- or polyfunctional starter molecules. Polyether polyols based on at least partial addition of ethylene oxide onto di- or polyfunctional starter molecules can also be employed as component A4) (nonionic hydrophilicizing agent).

Examples of suitable starter molecules which can be employed are water, butyldiglycol, glycerol, diethylene glycol, trimethyolpropane, propylene glycol, sorbitol, ethylenediamine, triethanolamine or 1,4-butanediol. Preferred starter molecules are water, ethylene glycol, propylene glycol, 1,4-butanediol, diethylene glycol and butyldiglycol.

Particularly preferred embodiments of the polyurethane dispersions (I) comprise as component A2) a mixture of polycarbonate polyols and polytetramethylene glycol polyols, in which case the proportion in this mixture of polycarbonate polyols is ≧20% by weight to ≦80% by weight and the proportion of polytetramethylene glycol polyols is ≧20% by weight to ≦80% by weight in the mixture. A proportion of from ≧30% by weight to ≦75% by weight of polytetramethylene glycol polyols and a proportion of from ≧25% by weight to ≦70% by weight of polycarbonate polyols is preferred. A proportion of from ≧35% by weight to ≦70% by weight of polytetramethylene glycol polyols and a proportion of from ≧30% by weight to ≦65% by weight of polycarbonate polyols is particularly preferred, in each case with the proviso that the total of the percentages by weight of the polycarbonate polyols and polytetramethylene glycol polyols is ≦100% by weight and the proportion of the total of polycarbonate polyols and polytetramethylene glycol polyether polyols in component A2) is ≧50% by weight, preferably ≧60% by weight and particularly preferably ≧70% by weight.

Isocyanate-reactive anionic or potentially anionic hydrophilicizing agents of component B1) mean all compounds having at least one isocyanate-reactive group such as an amino, hydroxy or thiol group, and at least one functionality such as, for example, —COO⁻M⁺, —SO₃⁻M⁺, —PO(O⁻M⁺)₂ with M⁺ for example equal to metal cation, H⁺, NH₄⁺, NHR₃⁺, where R may in each case be a C₁—C₁₂-alkyl radical, C₅—C₆-cycloalkyl radical and/or a C₂—C₄-hydroxyalkyl radical, which on interaction with aqueous media is involved in a pH-dependent dissociation equilibrium and may in this way have a negative or neutral charge.

The isocyanate-reactive anionic or potentially anionic hydrophilicizing agents are preferably isocyanate-reactive amino-functional anionic or potentially anionic hydrophilicizing agents.

Suitable anionically or potentially anionically hydrophilicizing compounds are monoamino and diamino carboxylic acids, monoamino and diamino sulphonic acids, and monoamino and diamino phosphonic acids and salts thereof. Examples of such anionic or potentially anionic hydrophilicizing agents are N-(2-aminoethyl)-β-alanine, 2-(2-aminoethylamino)ethanesulphonic acid, ethylenediaminepropyl- or -butylsulphonic acid, 1,2- or 1,3-propylenediamine-β-ethylsulphonic acid, glycine, alanine, taurine, lysine, 3,5-diaminobenzoic acid and the adduct of IPDA and acrylic acid (EP-A 0 916 647, Example 1). A further possibility is to use cyclohexylaminopropanesulphonic acid (CAPS) from WO-A 01/88006 as anionic or potentially anionic hydrophilicizing agent.

Preferred anionic or potentially anionic hydrophilicizing agents of component B1) are those of the aforementioned type having carboxylate or carboxylic acid groups and/or sulphonate groups, such as the salts of N-(2-aminoethyl)-β-alanine, of 2-(2-aminoethylamino)ethanesulphonic acid or of the adduct of IPDA and acrylic acid (EP-A 0 916 647, Example 1).

It is also possible to use mixtures of anionic or potentially anionic hydrophilicizing agents and nonionic hydrophilicizing agents for the hydrophilicizing.

In a further embodiment of the process of the invention, the reaction mixture in step A) further includes:
A3) hydroxy-functional compounds having molecular weights of from ≧62 g/mol to ≦399 g/mol.

The compounds of component A3) have molecular weights of from ≧62 g/mol to ≦399 g/mol.

It is possible to employ in A3) polyols of the said molecular weight range having up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and any mixtures thereof with one another.

Also suitable are ester diols of the said molecular weight range such as α-hydroxybutyl-ε-hydroxycaproic acid esters, ω-hydroxyhexyl-γ-hydroxybutyric acid esters, adipic acid (β-hydroxyethyl) ester or terephthalic acid bis(β-hydroxyethyl) ester.

It is also possible in addition to employ in A3) monofunctional, isocyanate-reactive compounds containing hydroxyl groups. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds of component A3) are 1,6-hexanediol, 1,4-butanediol, neopentyl glycol and trimethylolpropane.

In a further embodiment of the process of the invention, the reaction mixture in step A) further includes:
A4) isocyanate-reactive, anionic or potentially anionic and, where appropriate, nonionic hydrophilicizing agents.

Anionically or potentially anionically hydrophilicizing compounds of component A4) mean all compounds having at least one isocyanate-reactive group such as an amino, hydroxy or thiol group, and at least one functionality such as, for example, —COO⁻M⁺, —SO₃⁻M⁺, —PO(O⁻M⁺)₂ with M⁺ for example equal to metal cation, H⁺, NH₄⁺, NHR₃⁺, where R may in each case be a C₁—C₁₂-alkyl radical, C₅—C₆-cycloalkyl radical and/or a C₂—C₄-hydroxyalkyl radical, which on interaction with aqueous media is involved in a pH-dependent dissociation equilibrium and may in this way have a negative or neutral charge. Suitable anionically or potentially anionically hydrophilicizing compounds are for example monohydroxy and dihydroxy carboxylic acids, monohydroxy and dihydroxy sulphonic acids, and monohydroxy and dihydroxy phosphonic acids and salts thereof. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as described in DE-A 2 446 440, page 5-9, formula I-III. Preferred anionic or potentially anionic hydrophilicizing agents of component A4) are those of the aforementioned type having carboxylate or carboxylic acid groups and/or sulphonate groups.

Particularly preferred anionic or potentially anionic hydrophilicizing agents are those comprising carboxylate or carboxylic acid groups as ionic or potentially ionic groups, such as dimethylolpropionic acid, dimethylolbutyric acid and hydroxypivalic acid and/or salts thereof.

Suitable nonionically hydrophilicizing compounds of component A4) are for example polyoxyalkylene ethers comprising at least one hydroxy or amino group, preferably at least one hydroxy group. Examples thereof are the monohydroxy-functional polyalkylene oxide polyether alcohols having a statistical average of from ≧5 to ≦70, preferably ≧7 to ≦55 ethylene oxide units per molecule and as are obtainable by alkoxylation of suitable starter molecules. These are either pure polyethylene oxide ethers or mixed polyalkylene oxide ethers, in which case they comprise ≧30 mol %, preferably ≧40 mol %, based on all the alkylene oxide units present, of ethylene oxide units.

Preferred polyethylene oxide ethers of the aforementioned type are monofunctional mixed polyalkylene oxide polyethers having ≧40 mol % to ≦100 mol % of ethylene oxide units and ≧0 mol % to ≦60 mol % of propylene oxide units.

Preferred nonionically hydrophilicizing compounds of component A4) are those of the aforementioned type, being block (co)polymers prepared by blockwise addition of alkylene oxides onto suitable starters.

Suitable starter molecules for such nonionic hydrophilicizing agents are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers such as, for example, diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anisic alcohol or cinnamic alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the aforementioned type. Diethylene glycol monobutyl ether or n-butanol are particularly preferably used as starter molecules.

Alkylene oxides suitable for the alkoxylation reaction are in particular ethylene oxide and propylene oxide, which can be employed in any sequence or else in a mixture in the alkoxylation reaction.

In a further embodiment of the process of the invention, the free NCO groups of the prepolymers are further wholly or partly reacted in step B) with
B2) amino-functional compounds having molecular weights of from ≧32 g/mol to 400 g/mol.

It is possible to employ as component B2) di- or polyamines such as 1,2-ethylenediamine, 1,2- and 1,3-diaminopropane, 1,4-diaminobutane, 1,6-diaminohexane, isophoronediamine, isomer mixtures of 2,2,4- and 2,4,4-trimethylhexamethylenediamine, 2-methylpentamethylenediamine, diethylenetriamine, triaminononane, 1,3- and 1,4-xylylenediamine, α,α,α′,α′-tetramethyl-1,3- and -1,4-xylylenediamine and 4,4-diaminodicyclohexylmethane and/or dimethylethylenediamine. It is likewise possible, but less preferred, to use hydrazine and hydrazides such as adipohydrazide.

It is additionally possible to employ as component B2) also compounds which, besides a primary amino group, also have secondary amino groups or, besides an amino group (primary or secondary), also have OH groups. Examples thereof are primary/secondary amines such as diethanolamine, 3-amino-l-methylaminopropane, 3-amino-l-ethylaminopropane, 3-amino-l-cyclo-hexylaminopropane, 3-amino-l-methylaminobutane, alkanolamines such as N-aminoethyl-ethanolamine, ethanolamine, 3-aminopropanol, neopentanolamine.

It is further possible to employ as component B2) also monofunctional isocyanate-reactive amine compounds such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine or suitable substituted derivatives thereof, amide amines from diprimary amines and monocarboxylic acids, monoketimes of diprimary amines, primary/tertiary amines such as N,N-dimethylaminopropylamine. Preferred compounds of component B2) are 1,2-ethylenediamine, 1,4-diaminobutane and isophoronediamine.

In a further embodiment of the process of the invention, component A1) in the preparation of the aqueous, anionically hydrophilicized polyurethane dispersions (I) is selected from the group comprising 1,6-hexamethylene diisocyanate, isophorone diisocyanate and/or the isomeric bis(4,4′-isocyanatocyclohexyl)methanes, and where moreover component A2) includes a mixture of polycarbonate polyols and polytetramethylene glycol polyols, where the proportion of the total of the polycarbonate polyols and of the polytetramethylene glycol polyether polyols in component A2) is ≧70% by weight to ≦100% by weight.

Besides the polyurethane dispersions (I) and the additives it is also possible to use further auxiliaries.

Examples of such auxiliaries are thickeners or thixotropic agents, antioxidants, light stabilizers, emulsifiers, plasticizers, pigments, fillers and/or flow control agents.

Thickeners which can be employed are commercially available thickeners such as dextrin derivatives, starch derivatives or cellulose derivatives such as cellulose ethers or hydroxyethylcellulose, polysaccharide derivatives such as gum arabic or guar, organic completely synthetic thickeners based on polyacrylic acids, polyvinylpyrrolidones, poly(meth)acrylic compounds or polyurethanes (associative thickeners), and inorganic thickeners such as bentonites or silicas.

The compositions of the invention may in principle also comprise crosslinkers such as unblocked polyisocyanates, amide- and amine-formaldehyde resins, phenol resins, aldehyde and ketone resins, such as, for example, phenol-formaldehyde resins, resols, furan resins, urea resins, carbamic ester resins, triazine resins, melamine resins, benzoguanamine resins, cyanoamide resins or aniline resins.

In an exemplary formulation for preparing the polyurethane dispersions, components A1) to A4) and B1) to B2) are employed in the following amounts, with the individual amounts always adding up to ≦100% by weight:

≧5% by weight to ≦40% by weight of component A1);

≧55% by weight to ≦90% by weight of component A2);

≧0.5% by weight to ≦20% by weight total of components A3) and B2);

≧0.1% by weight to ≦25% by weight total of components A4) and B1), using ≧0.1% by weight to ≦5% by weight of anionic or potentially anionic hydrophilicizing agents from A4) and/or B1), based on the total amounts of components A1) to A4) and B1) to B2).

In a further exemplary formulation for preparing the polyurethane dispersions, components A1) to A4) and B1) to B2) are employed in the following amounts, with the individual amounts always adding up to ≦100% by weight:

≧5% by weight to ≦35% by weight of component A1);

≧60% by weight to ≦90% by weight of component A2);

≧0.5% by weight to ≦15% by weight total of components A3) and B2);

≧0.1% by weight to ≦15% by weight total of components A4) and B1), using ≧0.2% by weight to ≦4% by weight of anionic or potentially anionic hydrophilicizing agents from A4) and/or B 1), based on the total amounts of components A1) to A4) and B1) to B2).

In a very particularly preferred formulation for preparing the polyurethane dispersions, components A1) to A4) and B1) to B2) are employed in the following amounts, with the individual amounts always adding up to ≦100% by weight:

≧10% by weight to ≦30% by weight of component A1);

≧65% by weight to ≦85% by weight of component A2);

≧0.5% by weight to ≦14% by weight total of components A3) and B2);

≧0.1% by weight to ≦13.5% by weight total of components A4) and B1), using ≧0.5% by weight to ≦3.0% by weight of anionic or potentially anionic hydrophilicizing agents from A4) and/or B1), based on the total amounts of components A1) to A4) and B1) to B2).

Preparation of the anionically hydrophilicized polyurethane dispersions (I) can be carried out in one or more stage(s) in homogeneous or, in the case of multistage reaction, partly in disperse phase. Complete or partial polyaddition of A1) to A4) is followed by a dispersing, emulsifying or dissolving step. This is followed where appropriate by a further polyaddition or modification in disperse phase.

Examples of processes which can be used in this connection are prepolymer mixing processes, acetone processes or melt dispersing processes. The acetone process is preferably used.

For preparation by the acetone process, normally ingredients A2) to A4) and the polyisocyanate component A1) are initially introduced in whole or in part to prepare an isocyanate-functional polyurethane prepolymer and, where appropriate, are diluted with a water-miscible solvent which is inert to isocyanate groups, and heated to temperatures in the range from ≧50° C. to ≦120° C. To expedite the isocyanate addition reaction it is possible to employ catalysts known in polyurethane chemistry.

Suitable solvents are the usual aliphatic, keto-functional solvents such as acetone or 2-butanone, which can be added not only at the start of the preparation but, where appropriate, also in portions later. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can be employed in addition and be wholly or partly distilled out or, in the case of N-methylpyrrolidone, N-ethylpyrrolidone, remain completely in the dispersion. However, it is preferred to use no other solvents apart from the usual aliphatic, keto-functional solvents.

Subsequently, ingredients A1) to A4) which have where appropriate not been added at the start of the reaction are metered in.

In the preparation of the polyurethane prepolymers from A1) to A4), the amount of substance ratio of isocyanate groups to with isocyanate reactive groups is for example ≧1.05 to ≦3.5, preferably ≧1.2 to ≦3.0 and particularly preferably ≧1.3 to ≦2.5.

Reaction of components A1) to A4) to give the prepolymer takes place partly or completely, but preferably completely. Polyurethane prepolymers comprising free isocyanate groups are thus obtained undiluted or in solution.

In the neutralization step for partial or complete conversion of potentially anionic groups into anionic groups, bases such as tertiary amines, for example trialkylamines having ≧1 to ≦12, preferably ≧1 to ≦6 C atoms, particularly preferably ≧2 to ≦3 C atoms in each alkyl radical or alkali metal bases such as the corresponding hydroxides are employed.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also for example have hydroxyl groups, as in the dialkylmonoalkanolamines, alkyldialkanolamines and trialkanolamines. It is also possible where appropriate to employ inorganic bases such as aqueous ammonia solution or sodium hydroxide or potassium hydroxide as neutralizing agents.

Ammonia, triethylamine, triethanolamine, dimethylethanolamine or diisopropylethylamine, and sodium hydroxide and potassium hydroxide are preferred, and sodium hydroxide and potassium hydroxide are particularly preferred.

The amount of substance of the bases is between ≧50 mol % and ≦125 mol %, preferably between ≧70 mol % and ≦100 mol % of the amount of substance of the acidic groups to be neutralized. The neutralization can also take place at the same time as the dispersing when the dispersing water already contains the neutralizing agent.

Subsequently, in a further process step, the resulting prepolymer is dissolved with the aid of aliphatic ketones such as acetone or 2-butanone, if this has not yet happened or only partly happened.

In the chain extension in stage B), NH₂- and/or NH-functional components are reacted partly or completely with the still remaining isocyanate groups of the prepolymer. The chain extension is preferably carried out before the dispersing in water.

For chain termination, normally amines B2) with an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines from diprimary amines and monocarboxylic acids, monoketimes of diprimary amines, primary/tertiary amines such as N,N-dimethyl-aminopropylamine are used.

If anionic or potentially anionic hydrophilicizing agents complying with the definition B1) having NH₂ or NH groups are employed for the partial or complete chain extension, the chain extension of the prepolymers preferably takes place before the dispersing.

The amine components B1) and B2) can where appropriate be employed in water- or solvent-diluted form in the process of the invention, singly or in mixtures, with any sequence of addition being possible in principle.

If water or organic solvents are used as diluents, then the diluent content in the component employed in B) for chain extension is preferably ≧70% by weight to ≦95% by weight.

The dispersing preferably takes place following the chain extension. For this purpose, the dissolved and chain-extended polyurethane polymer is introduced, where appropriate with strong shearing, such as, for example, vigorous agitation, either into the dispersing water, or conversely the dispersing water is stirred into the chain-extended polyurethane polymer solutions. It is preferred to add the water to the dissolved chain-extended polyurethane polymer.

The solvent still present in the dispersions after the dispersing step is normally subsequently removed by distillation. A removal even during the dispersing is likewise possible.

The residual content of organic solvents in the polyurethane dispersions (I) is typically ≦1.0% by weight, preferably ≦0.5% by weight, based on the complete dispersion.

The pH of the polyurethane dispersions (I) of the invention is typically ≦9.0, preferably ≦8.5, particularly preferably less than ≦8.0 and is very particularly preferably ≧6.0 to ≦7.5.

The solids content of the polyurethane dispersions (I) is preferably ≧40% by weight to ≦70% by weight, particularly preferably ≧50% by weight to ≦65% by weight, very particularly preferably ≧55% by weight to ≦65% by weight and in particular ≧60% by weight to ≦65% by weight.

Examples of compositions of the invention are detailed hereinafter, with the total of the data in % by weight assuming a value of ≦100% by weight. These compositions include, based on dry matter, typically ≧80 parts by weight to ≦99.5 parts by weight of dispersion (I), ≧0 parts by weight to ≦10 parts by weight of foaming aid, ≧0 parts by weight to ≦10 parts by weight of crosslinker and ≧0 parts by weight to ≦10 parts by weight of thickener.

These compositions of the invention preferably include, based on dry matter, ≧85 parts by weight to ≦97 parts by weight of dispersion (I), ≧0.5 parts by weight to ≦7 parts by weight of foaming aid, ≧0 parts by weight to ≦5 parts by weight of crosslinker and ≧0 parts by weight to ≦5 parts by weight of thickener.

These compositions of the invention particularly preferably include, based on dry matter, ≧89 parts by weight to ≦97 parts by weight of dispersion (I), ≧0.5 parts by weight to ≦6 parts by weight of foaming aid, ≧0 parts by weight to ≦4 parts by weight of crosslinker and ≧0 parts by weight to ≦4 parts by weight of thickener.

Examples of compositions of the invention which include ethylene oxide/propylene oxide block copolymers as foam stabilizers are detailed hereinafter. These compositions include, based on dry matter, ≧80 parts by weight to ≦99.9 parts by weight of dispersion (I) and ≧0.1 parts by weight to ≦20 parts by weight of the ethylene oxide/propylene oxide block copolymers. The compositions preferably include, based on dry matter, ≧85 parts by weight to ≦99.5 parts by weight of dispersion (I) and 0.5 to 15 parts by weight of the ethylene oxide/propylene oxide block copolymers. Particular preference is given in this connection to ≧90 parts by weight to ≦99 parts by weight of dispersion (I) and ≧1 part by weight to ≦10 parts by weight of the ethylene oxide/propylene oxide block copolymers, and very particular preference is given to ≧94 parts by weight to ≦99 parts by weight of dispersion (I) and ≧1 to ≦6 parts by weight of the ethylene oxide/propylene oxide block copolymers.

In the context of the present invention, the statement “parts by weight” means a relative proportion but not within the meaning of the statement of % by weight. Consequently, the numerical total of the proportions by weight may also assume values above 100.

Besides the components mentioned it is possible to employ in the compositions of the invention also further aqueous binders. Such aqueous binders may be composed for example of polyester, polyacrylate, polyepoxide or other polyurethane polymers. Combination with radiation-curable binders as described for example in EP-A-0 753 531 is also possible. A further possibility is also to employ other anionic or nonionic dispersions such as polyvinyl acetate, polyethylene, polystyrene, polybutadiene, polyvinyl chloride, polyacrylate and copolymer dispersions.

The foaming in the process of the invention takes place by mechanical agitation of the composition at high speeds, by shaking or by decompression of a blowing gas.

The mechanical foaming can take place with any mechanical agitating, mixing and dispersing techniques. Air is ordinarily introduced during this, but nitrogen and other gases can also be used for this purpose.

The resulting foam is applied during the foaming or immediately thereafter to a substrate or put into a mould and dried. Particularly suitable substrates are papers or sheets which make it possible easily to detach the wound dressing before being employed to cover an injured site.

The application can take place for example by pouring or knife application, but other techniques known per se are also possible. Multilayer application with intermediate drying steps is in principle also possible.

A satisfactory speed of drying of the foams is observed even at 20° C., so that drying is possible without problems on injured human or animal tissue. However, for faster drying and fixation of the foams, preferably temperatures above 30° C. are used. However, temperatures of 200° C., preferably 150° C., particularly preferably 130° C., should not be exceeded during the drying, because otherwise unwanted yellowing of the foams may occur. Two-stage or multistage drying is also possible.

The drying ordinarily takes place with use of heating and drying apparatuses known per se, such as (circulating air) drying ovens, hot air or IR radiators. Drying by passing the coated substrate over heated surfaces, for example rolls, is also possible.

The application and the drying can in each case be carried out discontinuously or continuously, but a wholly continuous process is preferred.

The polyurethane foams can before drying thereof typically foam densities of from ≧50 g/litre to ≦800 g/litre, preferably ≧100 g/litre to ≦500 g/litre, particularly preferably ≧100 g/litre to ≦250 g/litre (mass of all the starting materials [in g] based on the foam volume of one litre).

After drying of the foams they can have a microporous, at least partly open-cell structure with intercommunicating cells. The density of the dried foams in this connection is typically below 0.4 g/cm³, and is preferably less than 0.35 g/cm³, particularly preferably ≧0.01 g/cm³ to ≦0.3 g/cm³ and is very particularly preferably ≧0.1 g/cm³ to ≦0.3 g/cm³.

The invention further relates to a shaped article obtainable by a process of the invention, having a foam region and a compressed region, where the compressed region is obtained by compressing a foam layer, and where this foam layer and the foam region include a polyurethane material which is obtained by foaming and drying a composition including an aqueous, anionically hydrophilicized polyurethane dispersion (I). The shaped article is consequently in one-piece form in relation to the foam region and the compressed region.

In one embodiment of the shaped article, the compressed region has a density of from ≧1 g/cm³ to ≦1.8 g/cm3. The density may also be in a range from ≧1.2 g/cm³ to ≦1.6 g/cm³ or from ≧1.4 g/cm³ to ≦1.5 g/cm³.

In a further embodiment of the shaped article, the compressed region has a transparency such that the intensity of visible light after passing through the compressed region is ≧50% to ≦95% of the original intensity. As already stated, this makes it possible to carry out a visual check of the covered region. It is also possible for the intensity to be ≧60% to ≦90% or ≧70% to ≦80% of the original intensity. Visible light refers in this connection to light with a wavelength of between 380 nm and 780 nm. The transparency can be determined for example by using white light.

The invention further relates to the use of a shaped article according to the present invention as sport article, textile article, cosmetic article or wound dressing. The use as wound dressing is preferred. Advantageously, the wound dressing can be shaped so that it can be placed on parts of the body. One example of a part of the body is the heel, the forehead, the chin, the neck, the iliac crest or the buttocks. The part of the body may further be for example a joint. In terms of its size, the wound dressing is adapted to the receiving part of the body, such as the heel or a joint, i.e. for example a finger joint, an elbow joint, a knee joint or an ankle joint.

The present invention is explained by means of the following drawings. These show:

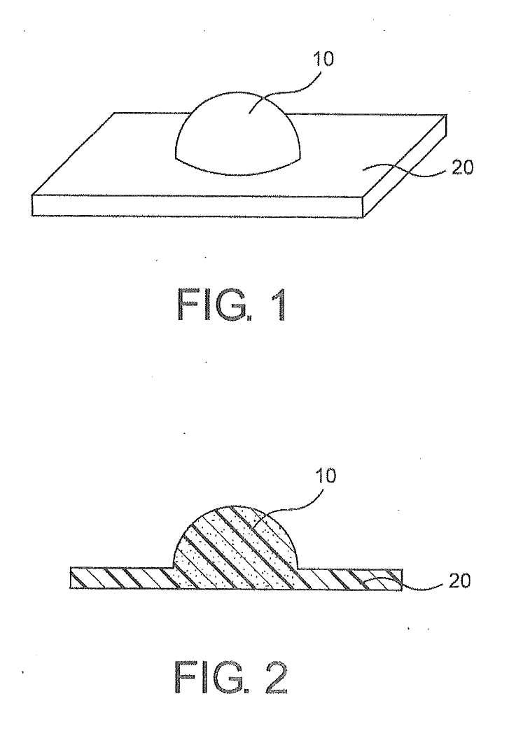

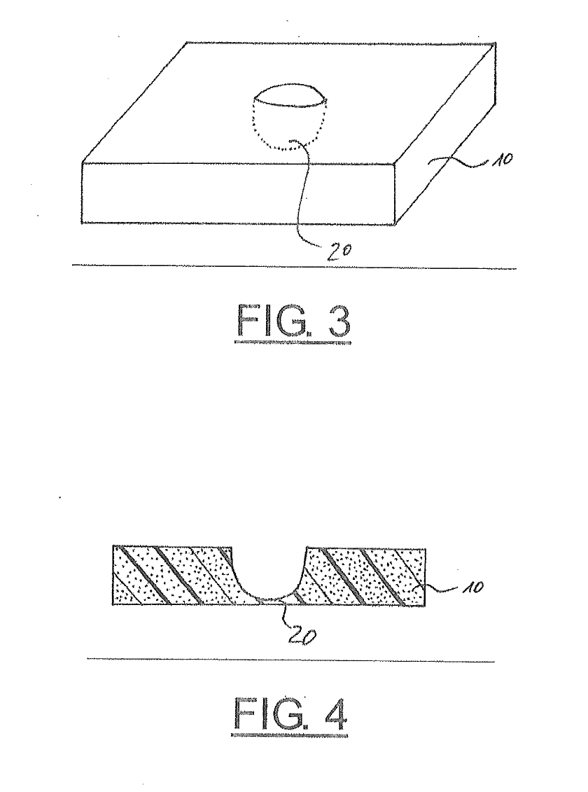

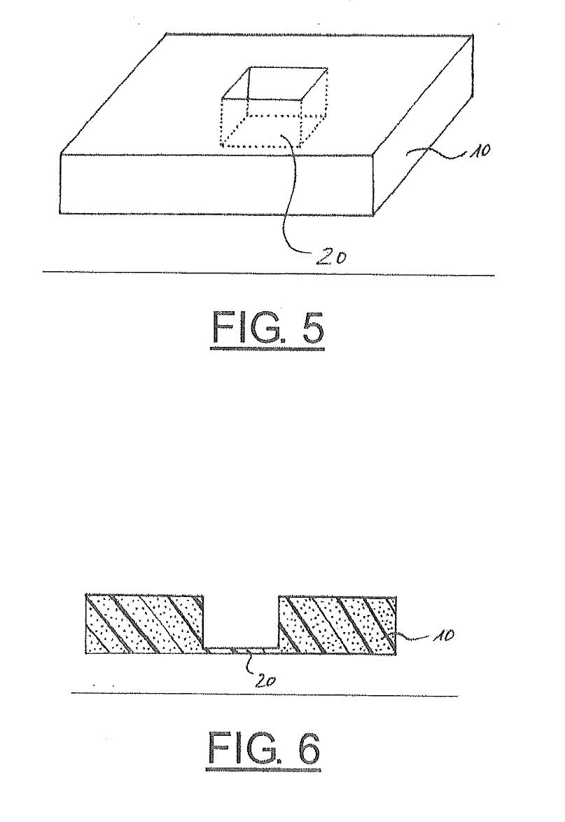

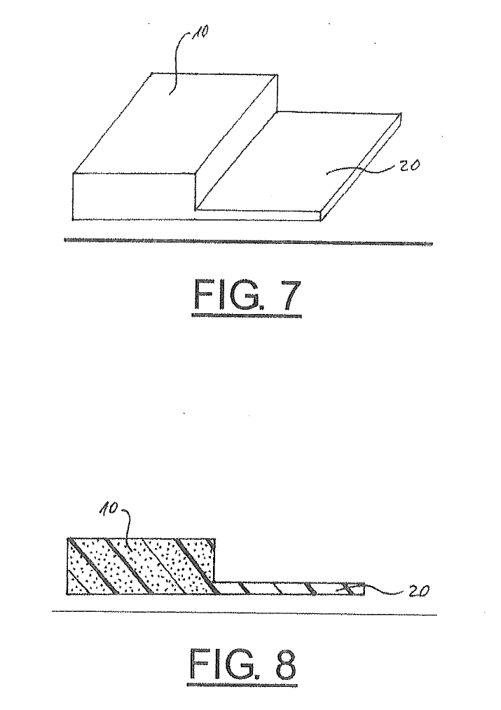

FIG. 1, FIG. 3, FIG. 5 and FIG. 7 in each case a perspective view of a shaped article produced according to the invention

FIG. 2, FIG. 4, FIG. 6 and FIG. 8 in each case a cross-sectional view of a shaped article produced according to the invention

FIG. 1, FIG. 3, FIG. 5 and FIG. 7 in each case show a perspective view of a shaped article produced according to the invention. The foam region 10 is located in the middle of the article in FIG. 1. The compressed region 20 is located around the foam region. In FIG. 3 and FIG. 5, the compressed region 20 is located in the middle of the article in each case, and the foam region 10 is located around the compressed region in each case. The foam region 10 and/or the compressed regions 20 in FIG. 1, FIG. 3 and/or FIG. 5 may also be located non-centrally in the article. The foam region 10 in FIG. 7 is located on the left-hand side of the article, the compressed region 20 on the right-hand side, the sides being interchangeable as desired. The articles are produced by compressing and heating a piece of the foam to be employed according to the invention with the dimensions of the finished article in a mould. The underside of the mould is flat and has no recesses or convex protrusions. The upper side of the mould is likewise flat, but has, for example, a circular or rectangular recess or convex protrusion at the desired position, e.g. in the middle. When the piece of foam is placed therebetween and compressed, the recess or convex protrusion leads to the foam region 10 being retained in the desired form, while the adjoining region is reshaped to the compressed region 20. The upper side of the mould may also be flat, and the underside of the mould has the abovementioned forms of shaping.

FIG. 2, FIG. 4, FIG. 6 and FIG. 8 in each case show a cross-sectional view of a shaped article produced according to the invention. In FIG. 2 this is the same article as in FIG. 1, FIG. 4 it is the same article as in FIG. 3, in FIG. 6 it is the same article as FIG. 5, and in FIG. 8 it is the same article as in FIG. 7. It is evident how the foam region 10 was retained as foam through the recess or convex protrusion in the upper side of the mould, while the adjoining region 20 has been compressed. The underside of the article has no gaps, but only compressed regions 20 or foam regions 10.

The foam regions 10 and/or compressed regions 20 may not only, as shown, have the shape of a cube or of a hemisphere, but may also possess any desired forms such as, for example, circular cylinders, more generally cylinders, prisms, tetrahedron, torus, cone, frustum or pyramid. It is also possible, moreover, to produce an article having a plurality of foam regions 10 and/or compressed regions 20, in which case the foam regions 10 and/or the compressed regions 20 may also have different geometries.

In one embodiment of the shaped article according to FIG. 1, the compressed region 20 has an area, based on the total area of the article, of ≧10% to ≦99%. The area preferably possesses a proportion of ≧20% to ≦80% or of ≧30% to ≦60%.

In one embodiment of the shaped article according to FIG. 3 or FIG. 5, the compressed region 20 has an area, based on the total area of the article, of ≧0.01% to ≦20%. The area preferably possesses a proportion of ≧0.1% to ≦15% or of ≧0.5% to ≦10%.

In one embodiment of the articles of the invention, the compressed region 20 has a layer thickness of ≧0.1 mm to ≦20 mm. The layer thickness may also be located in a range from ≧0.1 mm to ≦10 mm or from ≧0.1 mm to ≦5 mm or from ≧0.1 mm to ≦3 mm.

In one embodiment of the articles of the invention, the foam region 10 has a layer thickness of ≧1 mm to ≦30 mm. The layer thickness may also be located in a range from ≧2 mm to ≦20 mm or from ≧3 mm to ≦10 mm or from ≧3 mm to ≦8 mm

### Exemplary Embodiment

A polyurethane foam obtainable as described above and having a density of 180 kg/m³, corresponding to 0.18 g/cm³, and a thickness of 3.2 mm was thermoformed at a temperature of 160° C. and a pressure of 100 bar for a period of between 60 and 80 seconds to a thickness of 0.4 mm, i.e. 12.5% of the original thickness. A compact film was obtained.

1-13. (canceled)

14. A process for producing shaped articles comprising,
acting on a foam layer in a first region with a pressure of from 0 to 150 bar at a temperature of from 0° C. to less than or equal to 200° C., whereby the foam layer is compressed in the first region to greater than 12.5% to 100% of its original volume;
acting on the foam layer in a second region with a pressure of from 50 to 150 bar at a temperature of from 100° C. to 200° C., whereby the foam layer is compressed in the second region to greater than 0% to 12.5% of its original volume; and
wherein the foam layer comprises in the first region and the second region a polyurethane foam which is obtained by foaming and drying a composition comprising an aqueous, anionically hydrophilicized polyurethane dispersion.

15. The process according to claim 13, wherein the acting on the foam layer in the second region is carried out for a period of from 45 to 90 seconds.

16. The process according to claim 13, wherein the composition that forms the polyurethane foam further comprises additives which are selected from the group consisting of fatty acid amides, sulphosuccinamides, hydrocarbonsulphonates, hydrocarbon sulphates, fatty acid salts, alkyl polyglycosides, ethylene oxide/propylene oxide block copolymers, and mixtures thereof.

17. The process according to claim 16, wherein the ethylene oxide/propylene oxide block copolymers have a structure according to formula (1): 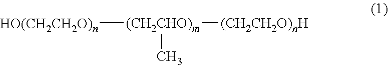
wherein the value for n is in a range of from 2 to 200, and the value for m is in a range of from 10 to 60.

18. The process according to claim 17, wherein the acting on the foam layer in the second region is carried out for a period of from 45 to 90 seconds.

19. The process according to claim 13, wherein the aqueous, anionically hydrophilicized polyurethane dispersion (I) is obtained by
A) providing an isocyanate-functional prepolymer having free NCO groups which is obtained from a reaction mixture comprising
A1) an organic polyisocyanate; and
A2) a polymeric polyol having number average molecular weights of from 400 to 8000 g/mol and OH functionalities of from 1.5 to 6
and wherein subsequently
B) the free NCO groups of the prepolymer are wholly or partly reacted with
B1) an isocyanate-reactive, anionic or potentially anionic hydrophilicizing agent, with chain extension, and
wherein the prepolymers are dispersed in water before, during or after B), and
wherein potentially ionic groups present in the reaction mixture are converted by partial or complete reaction with a neutralizing agent into the ionic form.

20. The process according to claim 19, wherein the reaction mixture in A) further comprises:
A3) a hydroxy-functional compound having a molecular weight of from 62 to 399 g/mol.

21. The process according to claim 19, wherein the reaction mixture in A) further comprises:
A4) an isocyanate-reactive, anionic or potentially anionic, or optionally nonionic hydrophilicizing agent.

22. The process according to claim 19, wherein the reaction mixture in A) further comprises:
A3) a hydroxy-functional compound having a molecular weight of from 62 to 399 g/mol; and
A4) an isocyanate-reactive, anionic or potentially anionic, or optionally nonionic hydrophilicizing agent.

23. The process according to claim 19, wherein the free NCO groups of the prepolymers in B) are further wholly or partly reacted with B2) an amino-functional compound having a molecular weight of from 32 to 400 g/mol.

24. The process according to claim 18, wherein component A1) is selected from the group consisting of 1,6-hexamethylene diisocyanate, isophorone diisocyanate, isomeric bis(4,4′-isocyanatocyclohexyl)methanes, and mixtures thereof; and
wherein component A2) comprises a mixture of polycarbonate polyols and polytetramethylene glycol polyols, wherein the proportion of the total of the polycarbonate polyols and of the polytetramethylene glycol polyether polyols in component A2) is from 70% to 100% by weight.

25. The process according to claim 24, wherein the reaction mixture in A) further comprises:
A3) a hydroxy-functional compound having a molecular weight of from 62 to 399 g/mol; and
A4) an isocyanate-reactive, anionic or potentially anionic, or optionally nonionic hydrophilicizing agent; and
wherein the free NCO groups of the prepolymers in B) are further wholly or partly reacted with B2) an amino-functional compound having a molecular weight of from 32 to 400 g/mol.

26. A shaped article obtained by the process according to claim 14, having a foam region and a compressed region, wherein the compressed region is obtained by compressing a foam layer, and wherein the foam layer and the foam region comprise a polyurethane material which is obtained by foaming and drying a composition comprising an aqueous, anionically hydrophilicized polyurethane dispersion.

27. The shaped article according to claim 26, wherein the compressed region has a density of from 1 to 1.8 g/cm³.

28. The shaped article according to claim 26, wherein the compressed region has a transparency such that the intensity of visible light after passing through the compressed region is from 50% to 95% of the original intensity.

29. A sport article, textile article, cosmetic article, or wound dressing comprising the shaped article according to claim 26.
